(19) United States
(12) Patent Application Publication
(10) Pub. No.: US 2022/0192469 A1
(43) Pub. Date: Jun. 23, 2022

(54) SURGICAL DELIVERY DEVICE AND METHOD

(71) Applicant: University College Dublin, National University of Ireland, Dublin, Dublin (IE)

(72) Inventors: Eoin O'CEARBHAILL, Dublin (IE); Ronan CAHILL, Dublin (IE); Paul HENEGHAN, Dublin (IE); Stefano DEOTTI, Dublin (IE)

(21) Appl. No.: 17/604,601

(22) PCT Filed: Apr. 20, 2020
(86) PCT No.: PCT/EP2020/061005

§ 371 Date: Oct. 18, 2021

(30) Foreign Application Priority Data
Apr. 18, 2019 (GB) 1905518.5

(51) Int. Cl. A61B 1/00; A61B 17/34; A61B 90/00

(52) U.S. Cl. CPC A61B 1/00087; A61B 2090/395; A61B 90/39; A61B 17/3478

(57) ABSTRACT

The present application relates to a surgical delivery device (10) suitable for submucosal dye delivery or for introduction or extraction of fluidic or gaseous material into or from tissue (T), the surgical delivery device comprising an elongate main body (12) defining an evacuatable chamber (18) internally thereof, a lateral window (20) in the body in communication with the chamber and a guideway (24a) extending through the body and into the chamber.

### FIELD OF THE INVENTION

The present invention relates to a surgical delivery device for affecting various functions such as, but not limited to, submucosal dye delivery, introduction or extraction of fluidic or gaseous material to tissue, and any other applicable procedure.

### BACKGROUND OF THE INVENTION

The invention addresses problems associated with the injection, extraction and/or relocation of fluids, solids and/or gases to a location below the tissue surface. The particular focus of the invention relates to endoscopic tattooing; the delivery of tattoo dye to the submucosa layer in the colon or intestine, but could also be used for many other applications for the delivery of other therapeutics below a layer of tissue, or for the extraction or relocation of fluid, solid or gas matter e.g. lesion margin identification and lymph node identification.

For surgical procedures requiring injection into the wall of the GI tract, considerable user-skill is required to control the depth of penetration and avoid leakage (too shallow an injection) and perforation (too deep an injection). There is also often a requirement for a second operator of the injection device. In the case of endoscopic tattooing, the aim is to inject tattoo dye into the submucosa. Currently, an exposed needle is used and the skill of the operator is required to locate the depth of the submucosa below the tissue. The aim of the current invention is to ensure material such as tattoo dye is injected consistently into the submucosa, and to make the procedure accessible to less skilled operators and to increase the speed and reliability of the procedure. Furthermore, as more expensive diagnostic dyes and therapeutics will soon be available, spillage/misplacement will have significant dosage and financial implications.

When performing polypectomy procedures such as EMR (endoscopic mucosal resection) or ESD (endoscopic submucosal dissection), the surgeon needs to quickly identify the site and margins of the lesion to be removed. After removal of the lesion, there might be need to operate on the site again to remove any residual cancerous tissue or lymph node metastases. To mark the location of a lesion, there are several options available which can be performed either at the time of initial lesion detection or later at its removal (through polypectomy or laparoscopic removal of a section of tissue). These include measuring the distance from the anus to the site of the lesion, intraoperative colonoscopy, endoscopic metal clips or endoscopic tattooing. Endoscopic tattooing marks the location of the lesion by injection of a dye into the submucosa. In order to enhance the quality of dye injection, prior saline injection to the submucosa has been proposed to decrease the likelihood of misdirected injection. There have been various dyes used for endoscopic tattooing.

Current procedures use an exposed needle (often referred to as endoscopic sclerotherapy needles) to try and pierce tissue down to the submucosal layer, with only manual control. The current invention avoids the use of an exposed needle, improving safety of the procedure. The invention also controls the depth of needle penetration, ensuring delivery of the needle to the appropriate location. In procedures like endoscopic tattooing, india ink leakage can lead to harmful complications such as abscess formation or peritonitis or create difficulties in surgical planning. It is anticipated that when higher commodity dyes and therapeutics are used in targeted injection for the management of colorectal disease, reliable methods to control the volume of dose injected will become of even more significance.

The present invention seeks to address the above mentioned problems.

### FIELD OF THE INVENTION

According to a first aspect of the present invention there is provided a surgical delivery device comprising an elongate main body defining an evacuatable chamber internally thereof; a lateral window in the body in communication with the chamber; and a guideway extending through the body and into the chamber.

Preferably, the guideway is shaped and dimensioned to permit the passage of a surgical instrument therealong.

Preferably, the guideway is shaped and dimensioned to permit the passage of a needle therealong.

Preferably, the guideway opens onto the chamber.

Preferably, the lateral window is shaped and dimensioned to permit a quantity of tissue to be drawn into the chamber via negative pressure applied to the chamber.

Preferably, the guideway comprises a proximal portion on one side of and terminating at the chamber, and a distal portion on an opposed side of and extending from the chamber.

Preferably, the proximal portion and the distal portion are coaxially aligned.

Preferably, the guideway comprises a cylindrical bore.

Preferably, the main body defines an evacuation circuit in communication with the chamber and operable to apply a negative pressure to the chamber when the lateral window is occluded.

Preferably, at least a portion of the evacuation circuit extends substantially parallel to the guideway.

Preferably, the chamber comprises a stop positioned to limit the depth to which tissue may be drawn into the chamber.

Preferably, the evacuation circuit extends around the stop to establish two or more regions within the chamber at which negative pressure may be applied.

Preferably, the main body comprises an atraumatic distal tip.

Preferably, the chamber is located adjacent the distal tip.

Preferably, the surgical delivery device comprises a needle captured for axial displacement within the guideway between a retracted position in which a tip of the needle is located in the guideway and an extended position in which the tip of the needle is located within or beyond the chamber.

Preferably, a tip of the needle is at least partially displaceable into the chamber.

Preferably, the tip of the needle is displaceable beyond the chamber.

Preferably, the tip of the needle is asymmetric.

Preferably, the needle comprises a lumen and an outlet providing fluid communication between the lumen and an exterior of the needle.

Preferably, the tip of the needle is sealed from the lumen.

Preferably, the outlet is located in a sidewall of the needle.

Preferably, the outlet is oriented towards the lateral window.

Preferably, the outlet is located adjacent the tip of the needle.

Preferably, the surgical delivery device comprises a positioning system adapted to maintain a particular orientation and/or to limit displacement of the needle as it undergoes reciprocal movement in the guideway.

Preferably, the positioning system is operable to prevent rotation of the needle about a longitudinal axis thereof.

Preferably, the alignment system is operable to stop the longitudinal displacement of the needle with the outlet located at a predetermined position within the chamber.

According to a second aspect of the present invention there is provided a method of delivering a substance to or from tissue, the method comprising the steps of locating a device having a main body defining an evacuatable chamber internally thereof and a lateral window in the body in communication with the chamber such that the window is located adjacent the target tissue; evacuating the chamber in order to draw the target tissue at least partially into the chamber; and delivering a substance into or out of the tissue while located in the chamber.

Preferably, the method comprises the step of advancing a surgical instrument into the chamber when the tissue has been drawn into the chamber such as to pierce the tissue; and delivering the substance through the surgical instrument.

Preferably, the method comprises the step of advancing the surgical instrument past the chamber in order to capture the target tissue thereon.

Preferably, the method comprises the step of evacuating the chamber through two or more channels.

Preferably, the method comprises the step of drawing the target tissue against a stop located within the chamber.

As used herein, the term “upstream” is a relative term intended to indicate a position or motion of one item or location relative to another item or location, in particular from a proximal position or location towards a distal position or location.

As used herein, the term “downstream” is a relative term intended to indicate a position or motion of one item or location relative to another item or location, in particular from a distal position or location back towards a proximal position or location.

As used herein, the term “substance delivery” is intended to mean the supply and/or withdrawal of a substance, for example a liquid or gas, but potentially also a solid and/or a combination thereof, to or from a target tissue, whether endoscopically, laparoscopically, subcutaneously or otherwise.

### BRIEF DESCRIPTION OF THE DRAWINGS

The present invention will now be described with reference to the accompanying drawings, in which;

FIG. 1 illustrates a sectioned side elevation of an endoscopic surgical device according to an embodiment of the present invention and located adjacent a target tissue;

FIG. 2 illustrates the endoscopic surgical device of FIG. 1 in which a chamber of the device is partially evacuated such as to draw the target tissue into the chamber;

FIG. 3 illustrates the endoscopic surgical device FIGS. 1 and 2 and having the target tissue drawn into the chamber and a needle forming part of the device being advanced into the target tissue;

FIG. 4 illustrates a modification to the method of operation shown in FIG. 3 whereby the needle is advanced beyond the chamber to secure the target tissue;

FIG. 5 illustrates a perspective view of an alignment system forming part of the endoscopic surgical device;

FIG. 6 illustrates a sectioned side elevation of the alignment system shown in FIG. 5;

FIG. 7 illustrates an end elevation of the alignment system;

FIG. 8 illustrates a perspective view of a further aspect of the alignment system;

FIG. 9 illustrates an end elevation of the arrangement of FIG. 8;

FIG. 10 illustrates a schematic sectioned side elevation of an alternative embodiment of a surgical delivery device according to the invention;

FIG. 11 illustrates the arrangement of FIG. 10 with a section of target tissue drawn into a chamber of the device and against a stop located within the chamber;

FIG. 12 illustrates the arrangement of FIGS. 10 and 11 and in which a needle forming part of the surgical delivery device has been advanced to skewer tissue within the chamber.

### DETAILED DESCRIPTION OF THE DRAWINGS

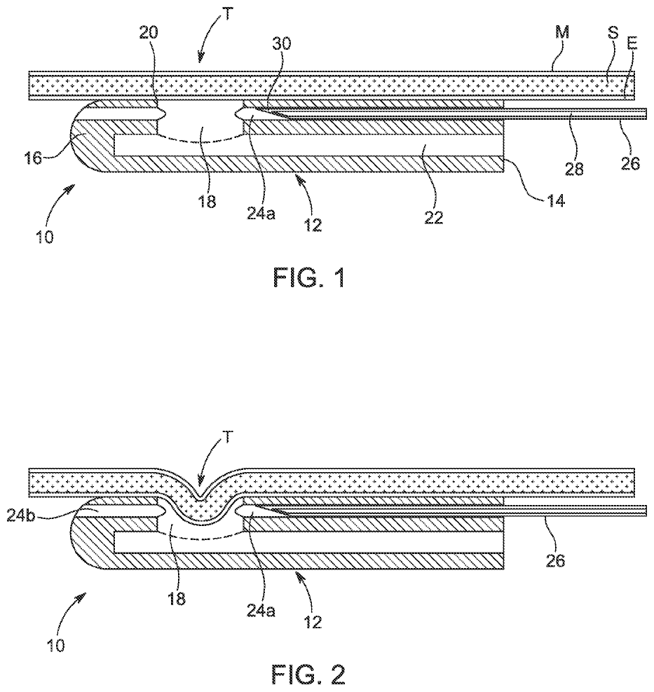

Referring now to FIGS. 1-5 of the accompanying drawings there is illustrated a surgical delivery device according to an embodiment of the present invention, generally indicated as 10, which is adapted to deliver a fluid, for example a tattoo dye (not shown) to a target region or portion of tissue T as described in detail hereinafter. It should however be understood that the device 10 and alternative embodiments thereof may be utilised or adapted to deliver other substances such as solids, liquids, gases or a combination thereof, in addition to being adapted to extract a sample of material from the target region or portion of tissue T or the like, for example performing a biopsy or the like. Additionally the delivery device 10 of the invention could be utilised to relocate material such as tissue to a different anatomical location.

The device 10 comprises a main body 12 which in the embodiment illustrated is elongate and cylindrical in form, such that it may pass through the lumen of a delivery conduit (not shown) or the like, and may be provided at or as the head of a steerable catheter (not shown) or the like in order to allow the delivery device 10 to be advanced into the desired position adjacent the target section of tissue T. The main body 12 comprises a proximal end 14 and a distal tip 16 which is preferably rounded or blunt in order to provide an atraumatic tip. The main body 12 defines a chamber 18 internally thereof and which is externally accessible via a lateral window 20 provided in a sidewall of the main body 12 on one side thereof. The chamber 18 is substantially cylindrical in the preferred embodiment, although any other suitable shape, volume and/or orientation may be employed. In the embodiment illustrated the chamber 18 and lateral window 20 are located adjacent the distal tip 16, although it will be appreciated that the position and orientation of the chamber 18 and window 20 may be varied as required, for example depending on the direction from which the surgical device 10 is to be advanced into position relative to the target tissue T.

The surgical delivery device 10 additionally comprises an evacuation circuit which in the embodiment illustrated is comprised primarily of an elongate channel 22 which extends longitudinally through the main body 12 from the proximal end 14 into fluid communication with the chamber 18 in order to allow the chamber 18 to be evacuated via the channel 22, by applying a negative pressure through the channel 22 via any suitable vacuum pump (not shown) or the like which may be connected to the channel 22 in conventional fashion. It will of course be understood that the channel 22 could be partially or fully located externally of the main body 12 but this would be a less preferred design choice in particular where the delivery device 10 is to be passed through a catheter or the like. In additional any other suitable functional means of effecting evacuation of the chamber 18 may be employed in place of the channel 22.

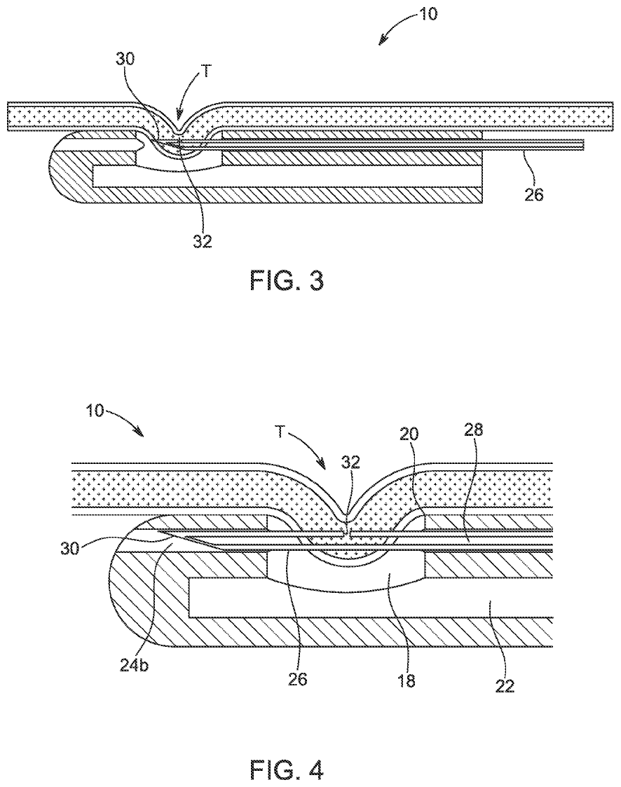

As will be described in greater detail hereinafter, the surgical delivery device 10 may thus be positioned adjacent the target tissue T such that the lateral window 20 overlies at least a portion of the target tissue T, which thereby effectively occludes the lateral window 20. Thus once negative pressure is applied to the chamber 18 via the channel 22 the target tissue T will be drawn into the chamber 18 as illustrated in FIGS. 2 and 3. The negative pressure may be maintained until the desired surgical procedure to be effected is complete, or may be released earlier if appropriate.

The surgical delivery device 10 further comprises a guideway 24 which, in the embodiment illustrated, runs essentially parallel to the channel 22 from the proximal end 14 into the chamber 18, and preferably extends beyond the chamber 18 to or close to the distal tip 16. The guideway 24 thus comprises a proximal portion 24a which is located upstream of the chamber 18 and a distal portion 24b which is located downstream of the chamber 18, relative to movement through the guideway 24 from the proximal end 14 towards the distal tip 16. The proximal portion 24a and distal portion 24b are axially aligned with one another and are preferably of the same cross section and dimension, although this is not essential.

The surgical delivery device 10 additionally comprises a surgical instrument which in the embodiment illustrated is in the form of a needle 26 which is captured in and slidable longitudinally along the guideway 24 as described hereinafter. The needle 26 is hollow and thus defines a lumen 28 therein, while also comprising a sealed and pointed tip 30 which in the embodiment illustrated is an asymmetric tip but may be of any other style or configuration. The needle 26 additionally comprises an opening or aperture 32 in a sidewall of the needle 26 adjacent the tip 30 and which thus allows fluid communication between the lumen 28 and the exterior of the needle 26. The needle 26 is reciprocally displaceable within the guideway 24 from a retracted position as illustrated in FIGS. 1 and 2 in which at least the needle tip 30 and adjacent portion of the needle 26 are located in the proximal portion 24a of the guideway 24, and an extended position as illustrated in FIG. 3 in which the tip 30 is advanced into the chamber 18, and more particularly such that the aperture 32 is located within the chamber 18 for the reasons described hereinafter. Depending on the design and dimensions of the needle 26, and in particular the tip 30, the distal portion 24b of the guideway 24 can serve to at least partially accommodate the tip 30 when the needle 26 is advanced to position the aperture 32 within the chamber 18, as the tip 30 might otherwise impact the sidewall of the chamber 18 thereby blunting the needle tip 30 over time.

Thus in use with the surgical delivery device 10 located adjacent the target tissue T, which in the embodiment illustrated comprises a layer of muscle M, submucosa S and the epithelium E, negative pressure is applied to the chamber 18 via the channel 22, thereby drawing the portion of target tissue T into the chamber 18 as illustrated in FIG. 2. During this initial evacuation phase the needle 26 is in the retracted position in which the tip 30 is located downstream of the chamber 18 within the proximal portion 24a.

Once the target tissue T is contained within the chamber 18 the needle 26 is advanced such that the tip 30 enters the chamber 18 and will therefore pierce the target tissue T, the needle 26 being advanced until the aperture 32 is located internally of the target tissue T, and in the embodiment illustrated is located within the submucosa S. At this point a substance, in particular a fluid and most particularly a tattoo dye, can be delivered to the submucosa S via the lumen 28 and aperture 32. It will of course also be appreciated that the aperture 32 and lumen 28 could be used to withdraw a substance from within the tissue T, effectively delivering a substance away from the target tissue T.

Once a suitable quantity of the dye or other substance has been delivered to the tissue T the needle 26 can be withdrawn into the retracted position, at which point the negative pressure can be released from the chamber 18 allowing the tissue T to be released from the device 10. The device 10 can then be withdrawn by any suitable means, or may be repositioned to another site to delivery further quantities of the dye or other substance.

Referring to FIG. 4, as a modification to the above methodology, the aperture 32 may be positioned longitudinally further back from the tip 30 of the needle 26, and when the target tissue T is then drawn into the chamber 18 under negative pressure the needle 26 can be advanced from the retracted position such that the tip 30 enters the tissue T and passes through the tissue T and continue upstream of the chamber 18, to enter into the distal portion 24b of the guideway 24. In this way the portion of target tissue T is effectively skewered by the needle 26, and it is then possible to release the negative pressure from the chamber 18 and the tissue T will remain captured on the needle 26. The dye or other substance can then be delivered into or away from the target tissue T as hereinbefore described, before again withdrawing the needle 26 into the retracted position to release the tissue T.

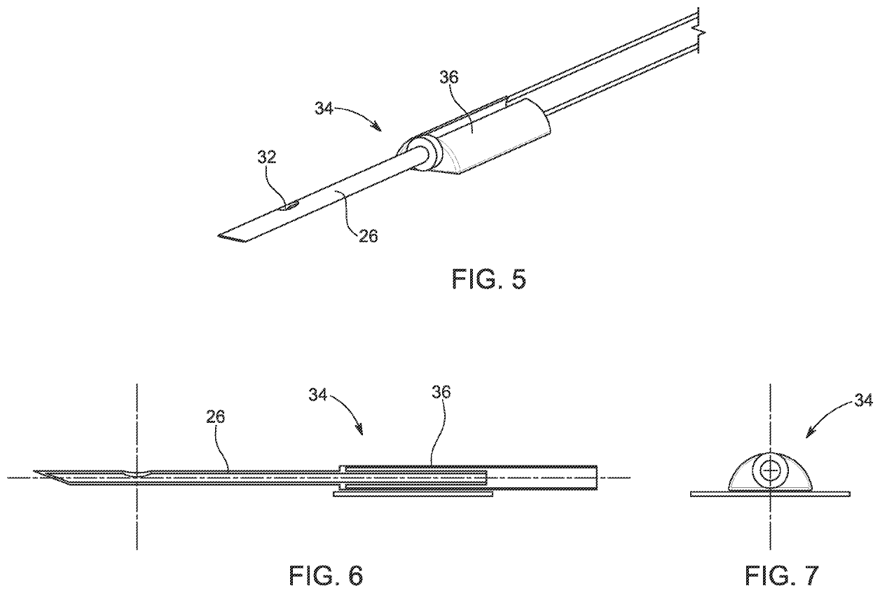

Referring now to FIGS. 5 to 9 there is illustrated an alignment system 34 which optionally but preferably forms part of the surgical delivery device 10, and is operable as hereinafter described to maintain a particular angular orientation of the needle 26 and/or limit the range of axial displacement that the needle 26 can undergo along the guideway 24. The alignment system 34 comprises collar 36 within which a rear portion of the needle 26 is captured in order to prevent the needle from undergoing any angular rotation about a longitudinal axis thereof, principally to ensure that the aperture 32 remains in a particular orientation, and preferably facing towards the lateral window 20 when the aperture 32 of the needle 26 is located within the chamber 18. This ensures that delivery of the dye or other substance via the aperture 32 is injected into the submucosa S and at a position which will improve penetration and dispersion of the dye into the tissue T. It is also the provision of the aperture 32 in the sidewall of the needle 26, as opposed to at the tip 30, which permits this functionality of improved penetration and dispersion by delivering the dye into the correct location with the target tissue T. It is also for this reason that the tip 30 of the needle 26 is sealed in order to ensure that the dye or other substance is only injected via the aperture 32.

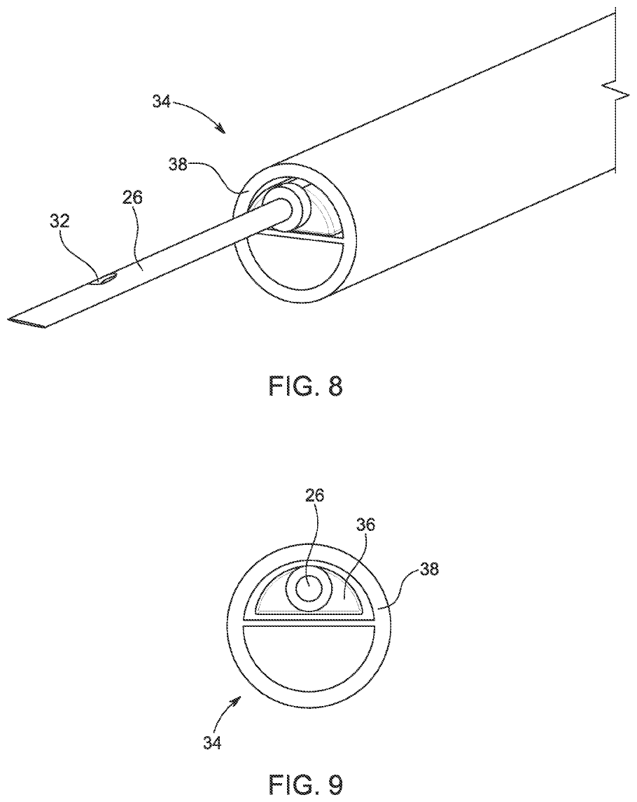

The alignment system 34 may additionally comprise a dual lumen tube 38 within which the collar 36 and captured portion of the needle 26 are located as illustrated in FIGS. 8 and 9. The tube 38 may be dimensioned to pass along the guideway 24, or the collar 36 including the needle 26 may be reversibly extendible out of the dual lumen tube 38 in order to effect extension and retraction of the needle 26 as hereinbefore described. Suitable mechanical limiters (not shown) may be provided as part of the alignment system 34 in order to limit this axial displacement of the needle 26, in order to ensure that the needle 26 cannot be over extended when piercing the target tissue T in the chamber 18, and thereby ensuring that the aperture 32 is always located at the correct position, preferably centrally of the chamber 18. This limitation of the extension of the needle 26 is also the mechanism by which it may be ensured that the sharpened tip 30 does not need to be used as a means of arresting the extension of the needle 26, which would require contact of the tip 30 with a surface and which would ultimately blunt the tip 30.

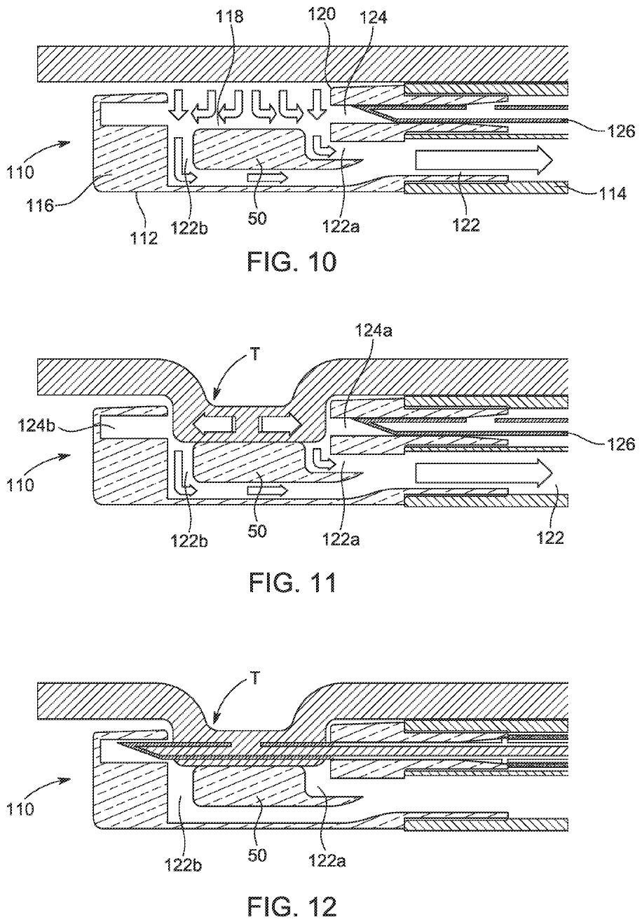

Referring now to FIGS. 10 to 12 there is illustrated a modified embodiment of a surgical delivery device according to the present invention, generally indicated as 110. In this modified embodiment like components have been accorded like reference numerals unless otherwise stated perform a like function.

The device 110 again comprises a main body 112 having a proximal end 114 and a distal tip 116, and defining a chamber 118 internally thereof which is open to the exterior of the device 110 via a lateral window 120. The device 110 comprises an evacuation circuit including a channel 122 which is provided in order to allow negative pressure to be applied to the chamber 118 during use. A guideway 124 is provided within the body 112 in order to permit a needle 126 to be advanced into and optionally past the chamber 118 as hereinbefore described with reference to the first embodiment.

Unlike the first embodiment the surgical delivery device 110 additionally comprises a stop 50 positioned within the chamber 118, or at the interface between the chamber 118 and the channel 122, which provides a dual functionality as hereinafter described. Firstly, the stop 50 effectively splits the evacuation channel 122 into two branches, a proximal branch 122a and a distal branch 122b, thereby creating a double suction force on the target tissue T from two spaced locations, while simultaneously limiting the depth to which the target tissue T may be drawn into the chamber 118, which combined with the double suction force achieves a more stable and even positioning of the tissue T within the chamber 118. This effectively creates a larger footprint of tissue T within the chamber 118, providing a greater length or volume of tissue T aligned with the needle 126 and into which the needle 126 may be advanced for delivering or extracting substances into or out of the tissue T. The guideway 124 may also comprise a proximal portion 124a and a distal portion 124b in order to allow the above described skewering of the target tissue by the needle 126, as illustrated in FIG. 12, thereby enabling the negative pressure to be released once the target tissue T has been skewered.

It will therefore be appreciated that the surgical delivery device of the invention overcomes various problems associated with the existing techniques and devices used for such procedures, in particular the problems of repeatability in relation to setting the depth of insertion of the needle; dependence on the skill of the clinician; the time taken to perform the procedure, which currently involves multiple potentially redundant steps; the requirement for an additional operator to actuate the needle to carry out the injection; and the complications which can arise, such as transmural injection, liquid spillage, involuntarily pinching which can occur.

The present invention provides improved safety whereby the design of the surgical delivery device 10; 110 prevents pinching of the target tissue, and the injection can only be preformed when the tissue is being drawn into the chamber of the device, which occurs only when suction has been previously activated by the operator, thereby decreasing the risk of involuntary pinching. The stable and even positioning of the tissue in the chamber provides a high level of control over the depth of insertion of the needle into the tissue, and the device of the invention allows repeatability of the procedure since the distance between the axis of the needle and the mechanical limit into the chamber of the device is constant, the injection therefore being performed to the same depth repeatedly.

1. A surgical delivery device comprising:
an elongate main body defining an evacuatable chamber internally thereof;
a lateral window in the body in communication with the chamber; and
a guideway extending through the body and into the chamber.

2. The surgical delivery device according to claim 1, in which the guideway is shaped and dimensioned to permit the passage of a surgical instrument therealong.

3. The surgical delivery device according to claim 1, in which the guideway is shaped and dimensioned to permit the passage of a needle therealong.

4. The surgical delivery device according to claim 1, in which the guideway opens onto the chamber.

5. The surgical delivery device according to claim 1, in which the lateral window is shaped and dimensioned to permit a quantity of tissue to be drawn into the chamber via negative pressure applied to the chamber from within the body.

6. The surgical delivery device according to claim 1, in which the guideway comprises a proximal portion on one side of and terminating at the chamber, and a distal portion on an opposed side of and extending from the chamber.

7. The surgical delivery device according to claim 6, in which the proximal portion and the distal portion are coaxially aligned.

8. The surgical delivery device according to claim 1, in which the guideway comprises a cylindrical bore.

9. The surgical delivery device according to claim 1, in which the main body defines an evacuation circuit in communication with the chamber and operable to apply a negative pressure to the chamber when the lateral window is occluded.

10. The surgical delivery device according to claim 9, in which at least a portion of the evacuation circuit extends substantially parallel to the guideway.

11. The surgical delivery device according to claim 1, in which the chamber comprises a stop positioned to limit the depth to which tissue may be drawn into the chamber.

12. The surgical delivery device according to claim 11, in which the evacuation circuit extends around the stop to establish two or more regions within the chamber at which negative pressure may be applied.

13. The surgical delivery device according to claim 1, in which the main body comprises an atraumatic distal tip.

14. The surgical delivery device according to claim 13, in which the chamber is located adjacent the distal tip.

15. The surgical delivery device according to claim 1, comprising a needle captured for axial displacement within the guideway between a retracted position in which a tip of the needle is located downstream of the chamber and an extended position in which the tip of the needle is located within or upstream of the chamber.

16. The surgical delivery device according to claim 15, in which the needle tip is at least partially displaceable into the chamber.

17. The surgical delivery device according to claim 15, in which the needle tip is displaceable beyond the chamber.

18. The surgical delivery device according to claim 15, in which the needle tip is asymmetric.

19. (canceled)

20. (canceled)

21. (canceled)

22. (canceled)

23. (canceled)

24. (canceled)

25. (canceled)

26. (canceled)

27. The method of delivering a substance to or from tissue, the method comprising the steps of:
locating a device having a main body;
defining an evacuatable chamber internally thereof and a lateral window in the body in communication with the chamber such that the window is located adjacent the target tissue;
evacuating the chamber in order to draw the target tissue at least partially into the chamber; and
delivering a substance into or out of the tissue while located in the chamber.

28. The method according to claim 27, further comprising the step of advancing a surgical instrument into the chamber when the tissue has been drawn into the chamber such as to pierce the tissue; and delivering the substance through the surgical instrument.

29. (canceled)

30. (canceled)

31. (canceled)
